(19) United States
(12) Patent Application Publication
(10) Pub. No.: US 2022/0215233 A1
(43) Pub. Date: Jul. 7, 2022

(54) End-to-End Deep Learning Approach to Predict Complex Stress and Strain Fields Directly from Microstructural Images

(71) Applicant: Massachusetts Institute of Technology, Cambridge, MA (US)

(72) Inventors: Markus J. Buehler, Boxford, MA (US); Chi Hua Yu, New Taipei City (TW); Zhenze Yang, Cambridge, MA (US)

(21) Appl. No.: 17/646,505

(22) Filed: Dec. 30, 2021

Related U.S. Application Data
(60) Provisional application No. 63/133,724, filed on Jan. 4, 2021.

(51) Int. Cl. G06N 3/04; G06N 3/08; G06T 11/00; G06F 30/20; B33Y 50/00; B29C 64/386

(52) U.S. Cl. CPC G06N 3/0454; G06N 3/088; B29C 64/386; G06F 30/20; B33Y 50/00; G06T 11/00

(57) ABSTRACT

Materials-by-design is a new paradigm to develop novel high-performance materials. However, finding materials with superior properties is often computationally or experimentally intractable because of the astronomical number of combinations in design spaces. The disclosure is a novel AI-based approach, implemented in a game-theory based generative adversarial neural network (GAN), to bridge the gap between the physical performance and design space. A end-to-end deep learning model predicts physical fields like stress or strain directly from the material geometry and microstructure. The model reaches an astonishing accuracy not only for predicted field data but also for secondary predictions, such as average residual stress at R₂˜0.96). Furthermore, the proposed approach offers extensibility by predicting complex materials behavior regardless of shapes, boundary conditions and geometrical hierarchy. The deep learning model demonstrates not only the robustness of predicting multi-physical fields, scalability, and extensibility. The disclosure may alter physical modeling and simulations by incorporating material geometry and boundary conditions into a graphical representation, and vastly improves the efficiency of evaluating physical properties of hierarchical materials directly from the geometry of its structural makeup.

### RELATED APPLICATION

This application claims the benefit of U.S. Provisional Application No. 63/133,724, filed on Jan. 4, 2021. The entire teachings of the above application are incorporated herein by reference.

### GOVERNMENT SUPPORT

This invention was made with government support under W911NF-19-2-0098 awarded by the Army Research Office (ARO), under Grant No. FA9550-15-1-0514 awarded by the Air Force Office of Scientific Research, and under Grant No. N00014-16-1-2333 awarded by the Office of Naval Research (ONR). The government has certain rights in the invention.

### BACKGROUND

Due to high demand for materials with superior mechanical properties and versatile functionalities, tuning composite designs is used in materials development. The essence of composites lies in introducing heterogeneity through combinations of multiple materials with distinct, often disparate, properties. Simple composites can be used in additive manufacturing (e.g., 3D printing).

### SUMMARY

Vastly enhanced mechanical performance can be demonstrated in modem ceramics matrix materials, fiber-reinforced polymers, or architected materials that include biologically evolved materials and bio-inspired materials by optimizing the spatial distributions of composite constituents. However, traditional manufacturing methods limit tunable composite designs because of difficulties conjoining different base materials and manipulating complex microstructures. To address these issues, additive manufacturing enables the production of composites with complex geometries.

Calculating physical properties of composites often relies on multiscale modeling approaches such as finite element modeling (FEM) or molecular dynamics (MD) simulations.

However, the design space of composites usually exists of an intractable number of combinations that hinders searching optimal designs either via experimental measurements or computational simulations. In some artificial intelligence (AI) methods, instead of using a brute-force approach, machine learning methods implement optimization of composite designs based on the calculation of FEM for various loading conditions, such as tensile or shear loading. However, these methods do not predict physical fields, and do not directly connect material microstructure to performance. For many design applications and engineering analyses, access to physical fields like strain or stress tensor distributions is necessary. These types of physical data also improve the physical relevance of AI approaches and provide more mechanistic insights.

In recent years, machine learning (ML) methods, especially deep learning (DL), have revolutionized the perspective of designing materials, modeling physical phenomena, and predicting properties. DL methods developed for computer vision and natural language processing can segment biomedical images, design de nova proteins and generate molecular fingerprints. Within the field of computational materials science, the abundance of data enables a boom of ML applications from quantum scale up to macroscale. By incorporating field-based (density functional theory (DFT)), particle-based (MD) and continuum based (FEM) modeling, ML shed light on predictions of quantum interactions, molecular force fields and material mechanics. However, most of these models are limited by a minimal level of information included in the prediction, and difficulty in generalization.

The present disclosure presents methods and systems that overcome these challenges by translating material composition directly to strain or stress fields as physical fields contain integral information of material behaviors to bridge physics and designs. The material composition provided to the model is in the form of a simple image of the geometry and microstructure that fully encodes material composition and boundary conditions. The present methods and systems illustrate s that the physical relationship between composite geometries and strain or stress fields can be directly established, at high levels of accuracy and predictability.

Materials-by-design is a new paradigm to develop novel high-performance materials. However, finding materials with superior properties is often computationally or experimentally intractable because of the astronomical number of combinations in design space. The present disclosure is a novel AI-based approach, implemented in a game-theory based generative adversarial neural network (GAN), to bridge the gap between the physical performance and design space. An end-to-end deep learning model predicts physical fields (e.g., stress or strain) directly from the material geometry and microstructure. The model reaches an astonishing accuracy, not only for predicted field data, but also for secondary predictions, such as average residual stress at R₂˜0.96). Furthermore, the proposed approach offers extensibility by predicting complex materials behavior regardless of shapes, boundary conditions and geometrical hierarchy. The deep learning model demonstrates not only the robustness of predicting multi-physical fields but also tremendous scalability and extensibility. The approach reported here may alter performing physical modeling and simulations by incorporating material geometry and boundary conditions into a graphical representation, and vastly improves the efficiency of evaluating physical properties of hierarchical materials directly from the geometry of its structural makeup.

In embodiments, a method (and corresponding system and non-transitory computer readable medium) of the disclosure can include generating, at a generator neural network, a fake field image based on adding noise to an inputted geometry images of a composite. The method can further include determining, at a discriminator neural network, whether the fake image generated by the generator based on the generated geometry images represents a ground truth. The method can further include generating a field prediction of at least one of a global property of a material and local property of the material. The field prediction can be generated upon the discriminator neural network determining the fake image generated represent the ground truth. The field prediction can be a prediction of at least one of a stress field and a strain field of the generated geometry images.

In embodiments, the method includes repeating the generating and determining until the discriminator determines the fake image generated by the generator represents the ground truth.

In embodiments, the method includes comparing the field prediction of the composites to a field prediction of strain and stress field information generated using a finite element method (FEM) to determine the accuracy of the field prediction.

In embodiments, the geometry images encode material composition and boundary conditions.

In embodiments, the geometry image includes microstructure.

In embodiments, the geometry image includes brittle units and soft units, the brittle units and soft unit being mechanically distinct in the properties of elasticity and plasticity.

In embodiments, the method further includes, based on the field prediction, translating a three-dimensional model into an additive manufacturing model for three-dimensional printing.

In embodiments, the generator and discriminator form a machine learning network. The machine learning network is a generative adversarial network (GAN).

In embodiments, a method (and corresponding system and non-transitory computer readable medium) of the disclosure can include training a machine learning network having a generator and a discriminator. The training includes (a) generating, at the generator, field images having random noises added based on inputted geometric images. The generator has a training objective to increase an error rate of the discriminator. The training further includes (b) comparing, using the discriminator, the generated field images to real field images. Each comparison determines whether the field images from the generator are real or fake. The discriminator has a training objective to optimize a capacity of identifying fake images produced by the generator. In an embodiment, the machine learning network is trained when the discriminator and generator reach an equilibrium such that one of the component networks maintains its status regardless of the other component networks.

In embodiments, the method further includes randomly generating the geometry images of the composites to be inputted to the generator.

In embodiments, the method further includes analyzing the randomly generated geometry images of the composites using a finite element method (FEM) to obtain real strain and stress field information to provide to the discriminator.

In embodiments, the geometry images encode material composition and boundary conditions.

In embodiments, the geometry image includes microstructure.

In embodiments, the geometry image includes brittle units and soft units, the brittle units and soft unit being mechanically distinct in the properties of elasticity and plasticity.

In embodiments, the method further includes running the trained machine learning network by providing a particular geometry image to the machine learning network, thereby generating a field prediction of stress fields and strain field of the particular geometry image. The method further includes based on the field prediction, translating a three-dimensional model into an additive manufacturing model for three-dimensional printing.

In embodiments, a system includes a processor and a memory with computer code instructions stored thereon. The processor and the memory, with the computer code instructions, being configured to cause the system to train a machine learning network having a generator and a discriminator. The training is performed by generating, at the generator, field images having random noises added based on inputted geometric images. The generator has a training objective to increase an error rate of the discriminator. The training is further performed by comparing, using the discriminator, the generated field images to real field images. Each comparison can determine whether the field images from the generator are real or fake. The discriminator has a training objective to optimize a capacity of identifying fake images produced by the generator. The machine learning network is trained when the discriminator and generator reach an equilibrium, the equilibrium being one of the component networks maintaining its status regardless of the other component networks.

In embodiments, the instructions are further configured to cause the system to randomly generating the geometry images of the composites to be inputted to the generator.

In embodiments, the instructions are further configured to cause the system to analyzing the randomly generated geometry images of the composites using a finite element method (FEM) to obtain real strain and stress field information to provide to the discriminator.

In embodiments, the geometry images encode material composition and boundary conditions.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a block diagram illustrating an example embodiment of a method employed by the present disclosure.

FIG. 2A is a diagram illustrating an example of such an evaluation, with loading applied in the x-direction.

FIG. 2B is a set of results illustrating typical results of strain or stress fields predictions of the test dataset.

FIG. 3A is a diagram illustrating stress and strain fields as well as displacements (incorporated in the overall deformation of the geometry) that are captured in the prediction.

FIG. 3B is a graph illustrating the distributions of L2 norm of predicted contours are plotted against two reference contours, clean contours and random contours.

FIG. 3C is a diagram illustrating recoverability of FEM and ML methods.

FIG. 3D is a graph illustrating ranks of recoverability of 400 composites in a test data set.

FIG. 3E is a diagram illustrating the method being used for local, smaller-scale patterns.

FIG. 4A is a diagram illustrating the von Mises stress fields calculated by the FEM and the trained model show high similarity, as visualized in two example cases of triangle and hexagon.

FIG. 4B is a diagram illustrating different loading conditions.

FIG. 4C is a diagram illustrating an example of a 32×32 profile.

FIG. 4D illustrates a generated L2 norm map that is used instead of the L2 norm for comparing high-resolution field image.

FIGS. 5A-B are diagrams of the graphical representation of composites and the distribution of 2000 data considering the ratio of soft units.

FIG. 6 is a diagram illustrating two types of geometries having cracks and resulting stress field predictions.

FIG. 7 illustrates a computer network or similar digital processing environment in which embodiments of the present invention may be implemented.

FIG. 8 is a diagram of an example internal structure of a computer (e.g., client processor/device or server computers) in the computer system of FIG. 7.

### DETAILED DESCRIPTION

A description of example embodiments follows.

The disclosed novel deep-learning based approach provides a direct translation of the composite geometry to strain or stress fields, realized using a game-theoretic approach implemented as a Generative Adversarial Network. The neural networks are trained with a relatively small amount of data but reach astonishing accuracy, transferability and hence broad applicability. The model predicts multiple mechanical fields with the same trained model covering multiple aspects of materials behaviors. By extracting information from the field predictions, the model can identify top designs of recoverability exactly and capture scale-consistent local patterns. Therefore, the disclosed approach is a promising tool to accelerate discovery of optimal geometric designs for materials such as metamaterials and hierarchical materials.

By using soft materials, the model can predict analogous stress fields around cracks. The results agree with the knowledge of the patterns of stress fields around cracks even if the ratio of soft materials for these cases are extremely skewed in the data distribution. The predictions are consistent regardless of shapes and positions of cracks or sharp inclusions, can describe the evolution of fields as cracks propagate. As a result, the model could also be used for crack-related design problems such as crack-resistant materials, providing added evidence for the transferability of the method.

The model can capture mechanical behaviors of composites with various shapes, different loading conditions and complicated geometries. The model can predict mechanical behaviors from random geometries and open the gate to accelerate searching optimal designs of composites with multiple mechanical functionalities, from the bottom up. In that context, one of the extraordinary advantages of using predicted stress and strain fields is that fields contain comprehensive information for various design purposes. Thus, the approach offers a high level of efficiency of predicting physical properties and accelerate the design process based on a transferrable ML method. Furthermore, the model incorporates non-square representations, multiple loading conditions, cracks, and high-resolution geometries. The extensions enable covering tremendous applications of FEM with lower computational costs and investigate complex systems of interest. Moreover, the disclosed approach can also be directly applied to experimental images for training of the model, which underscores the transferability of the approach, and provides a novel way to combine bottom-up modeling with experimental data sources for predictive methods.

Combined with optimization methods, the model can speed up the discovery of optimal designs without heavy computational loads. Moreover, the proposed approach is a general method for geometry-to-field translation. A similar protocol as developed here could be applied to other areas in the sciences, such as density functional theory fields, fluid mechanical fields, or electromagnetism.

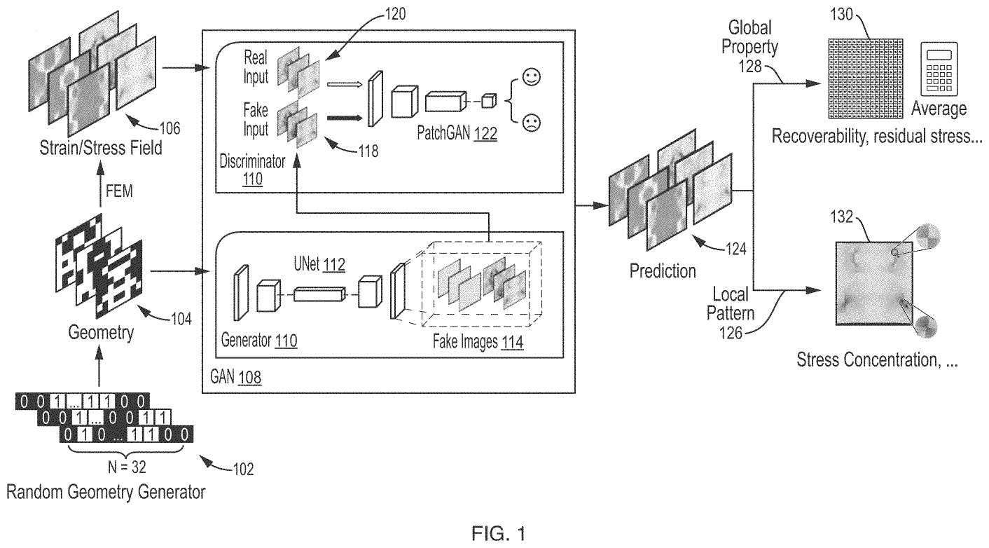

FIG. 1 is a block diagram 100 illustrating an example embodiment of a method employed by the present disclosure. Embodiments of the method employ a deep learning model implemented in a game-theory based Generative Adversarial Network approach (GAN) 108. This GAN model has two key components, Generator U-Net 110 and Discriminator PatchGAN 116. Geometric images 104 are generated by a random geometry generator 102.

The arrangement of block units is generated by a random geometry generator 102 to explore a broad range of arrangement combinations. For different geometries, strain or stress fields of composites under mechanical tests such as compression are obtained using FEM. The results from FEM are regarded as the ground truth for the purposes of training and evaluating the model. Both the geometry images and the strain or stress field images are collected to compose the dataset. In one example, the dataset consists of 2,000 data in total, which is split into a training dataset with 80% of the data and a test dataset with the rest 20% of the data.

The geometric images 104 are input to the generator 110 to generate field images of interest with random noise. The discriminator 116 evaluates these fake field images 114 generated by the generator 110 by comparing them with real field images 106 obtained from FEM. The fake field images 114 are based on the generated geometries but include incorrect/wrong fields (e.g., based on adding in random noise). The training objective of the generator 110 is to increase the error rate of the discriminator 116, while the discriminator 116 is trained to optimize the capacity of identifying fake field images 114 produced by the generator 114. Within the employed game theory framework, the GAN model 108 converges when the discriminator 116 and the generator 110 reach a Nash equilibrium, in which one component (e.g., one of the two models) maintains its status regardless of the actions of the opponent (e.g., the other of the two models). In essence, discriminator 116 analyzes real stress/strain fields 106 and fake stress/strain fields 114 based on the same geometry to determine which is a real input (e.g., fields 106) and which is a fake input (e.g., fields 114) from the generator 110. During training, the discriminator 116 improves to discern between fake (e.g., 114) and real (e.g., 106) stress and strain images, while the generator 110 improves to create better fake stress and strain images 114.

After training is complete, the model 108 can predict new field images 124 can be predicted bypassing conventional numerical simulations. The field predictions 124 can be further used to extract mechanical properties 126 and 128 of the composite given its geometry. As described below, not only global/mechanical properties 128 including stiffness and recoverability can be obtained, but also local properties/features/patterns 126 such as stress concentrations around cracks or sharp inclusions.

Composite profiles are illustrated with 8×8 graphical representations, but a person of ordinary skill in the art can recognize that other dimensions can be employed in other embodiments. Each composite profile for each a given geometry two different block units, a brittle unit and soft unit. Brittle units and soft units are mechanically distinct in elasticity and plasticity. A brute force method is executed to generate all possible combinations of two different units without fixing the composition. To simplify the geometry and shorten the calculation time in Abaqus, all composites created by the method are symmetric about Y-axis.

A FEM method generates the dataset using existing methods (e.g., a commercial ABAQUS/Explicit code (Dassault Systemes Simulia Corp., 2010). In the present method, strain or stress fields calculated by FEM are considered as the ground truth when comparing with ML results. All simulations are carried out in 2D. To include plastic deformation, crushable foam model with volumetric hardening embedded in Abaqus is implemented for composites. The method can output detailed material properties of two different units in composites are exhibited, as shown below in Table 1, which illustrates overall mechanical behavior of materials while the specific hardening curve is defined by Extended Data Table 2. To briefly summarize the difference of two materials shown by the data, the brittle unit has a relatively larger Young's modulus but a smaller yield strain compared with the soft unit.

EXTENDED DATA TABLE 1
Material Properties of Brittle and Soft Units

| Material behavior | Parameter | Brittle Unit | Soft Unit |
|---|---|---|---|
| Size | Length (Å) | 40 | 40 |
| Density | Mass density | 0.01173 | 0.00621 |
| | (10-5 kg/m3) | | |
| Elastic | Young's modulus | 66 | 13.9 |
| | (GPa) | | |
| | Poisson' ratio | 0.04 | 0.04 |
| Plastic | Compression yield | 1.5 | 1.1667 |
| (crushable foam) | stress ratio | | |
| | Hydrostatic yield | 4.5 | 5.8 |
| | stress ratio | | |
| | Foam hardening | Volumetric | Volumetric |

EXTENDED DATA TABLE 2
Foam hardening suboptions in ABAQUS 55 (Tabular
data of true stress and uniaxial plastic strain
in hardening stage) of soft and brittle units.

| | |
|---|---|
| Brittle unit | Soft unit |

| Order | YS (GPa) | UPS (%) | YS (GPa) | UPS (%) |
|---|---|---|---|---|
| 1 | 2.707 | 0.00 | 0.715 | 0.00 |
| 2 | 3.326 | 1.17 | 0.899 | 0.808 |
| 3 | 3.957 | 4.57 | 1.063 | 14.1 |
| 4 | 4.312 | 6.28 | 1.341 | 18.5 |
| 5 | 5.146 | 10.3 | 1.717 | 22.6 |
| 6 | 6.185 | 14.9 | 2.308 | 25.7 |

Once the material properties and geometry are defined, the composite is put under compressive loading and unloading for one cycle in Abaqus. Element “CPE4R” with a global size of six is used for composites during the calculation. In terms of boundary conditions, a middle line of the composite is set immobile along the x-axis direction due to the symmetry. The loading and unloading are realized by moving two symmetric analytical rigid shells. During the loading process, the magnitude of compressive strain is 10%. The contact type between composite and 2D shell is surface-to-surface contact using kinetic contact method. For either loading or unloading, 2D shell is moving in a time period of 500, consistent with Extended Data Table 1. Within the time period, the amplitude of displacement of the rigid shell follows “Smooth step” type embedded in Abaqus. After loading and unloading, images of strain or stress fields are postprocessed and collected for the dataset.

In one example embodiment, images of mechanical fields are generated first (e.g., by a program such as Abaqus Visualization) and then postprocessed (e.g., by code executing the described method written in python). As for generating the images of the mechanical fields, the images are plotted with both deformation, to capture the displacement field in the geometry change, and strain or stress field contours. The color spectrum for field contours is realized using a range for “white” having RGB=(216, 216, 216) as lower bound and a range for “red” having RGB=(216, 7, 0) for the upper bound. A person having ordinary skill in the art can recognize that lower and upper bound can vary in different embodiments but are consistent within one dataset. The same spectrum of bounds is also used for geometry input with “white” having RGB=(216, 216, 216) as a lower bound for brittle units and “red” having RGB=(216, 7, 0) as an upper bound for soft units. In example embodiments, the contour style used is “DISCRETE” and the number of intervals is set to 24. In example embodiments, for options for edges, “FEATURE” is used for visible edges and “THICK” is used specifically for green lines which represent analytical rigid bodies. Any triad, legend, title, state, annotation, compass and reference point are turned off to exclude useless information in the images. In addition, the background of the image is set to solid black (e.g., RGB=(0, 0, 0)). After all settings are applied, an image is generated with the “print” command in Abaqus.

Based on the images created (e.g., by Abaqus), a method (e.g., implemented by Python code) is executed to cut, resize and stitch images. The input image (e.g., geometry images) and target image (e.g., field images) are cut to keep the proportion of black background similar vertically and horizontally. The purpose of cutting operation is to make sure that the composites in the input image and target image are generally matching in shape to be read by GAN model. Once the images are cut, they are all resized to images having a common size, which in embodiments can be 512×512. After resizing, two images are stitched in accordance with the format of model input. In an embodiment, OpenCV52 is used for reading, cutting, plotting and stitching images.

To compare filed images, L2 norm is calculated to determine the difference between two images. Given two images P1 and P2, L2 norm between these two images are defined as:

$$Norm\_L2 = {\sum }_{i , j} {\vert P 1 ( i , j ) - P 2 ( i , j ) \vert}^{2}$$

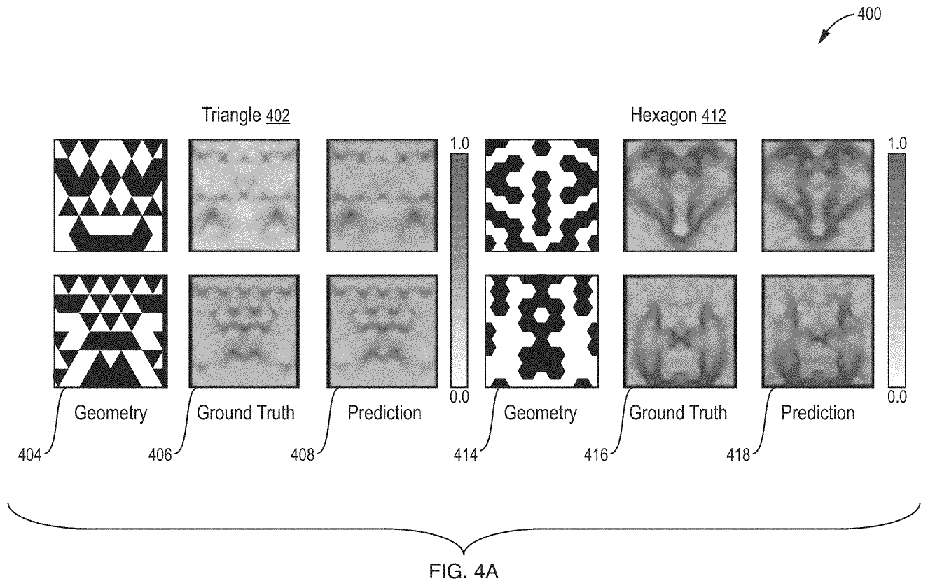

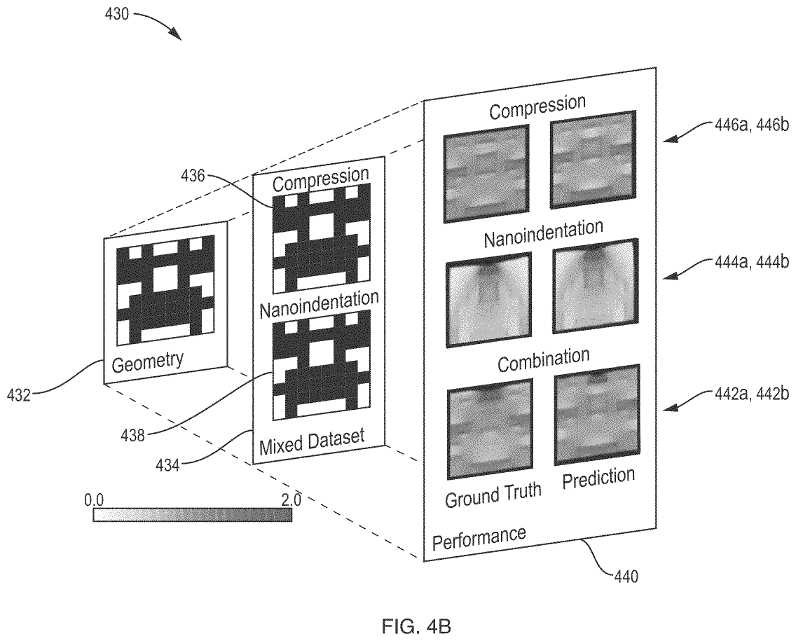

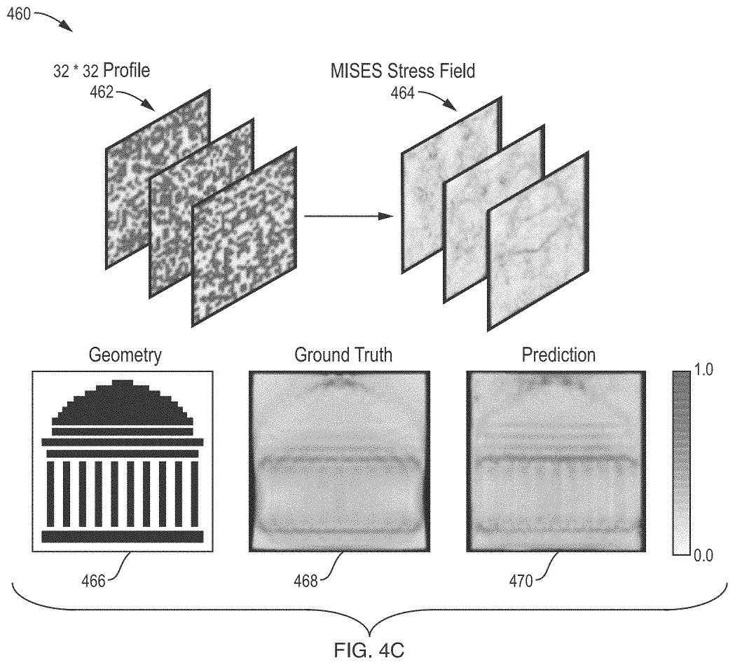

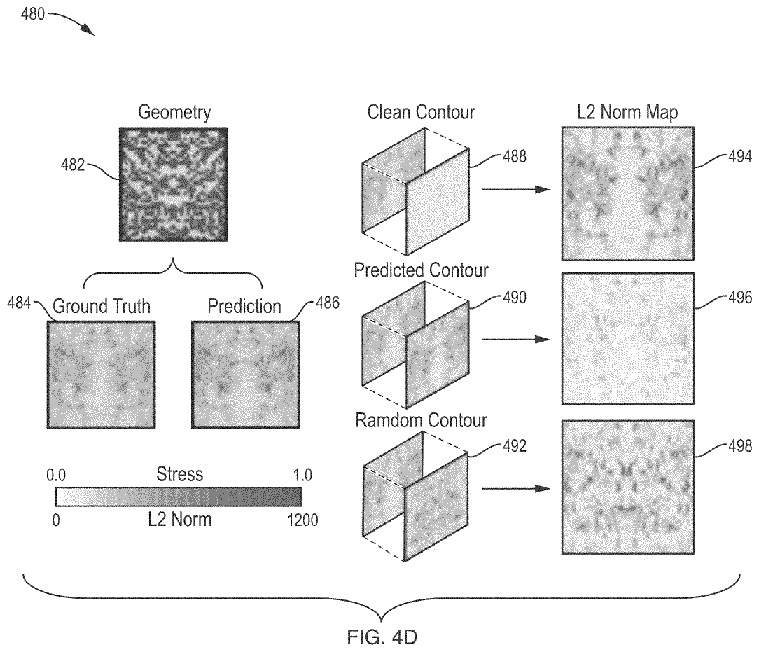

FIG. 4D illustrates a generated L2 norm map that is used instead of the L2 norm for comparing high-resolution field image. To derive a L2 norm map, the method first slices images of 512×512 into 128×128 patches, each with size of 4×4. Secondly, for each pair of corresponding patches in two images, L2 norm is calculated. The corresponding output is a matrix of 128×128 with each element corresponding to the difference between a pair of patches in two images. The matrix is named as L2 norm map 494, 496, and 498.

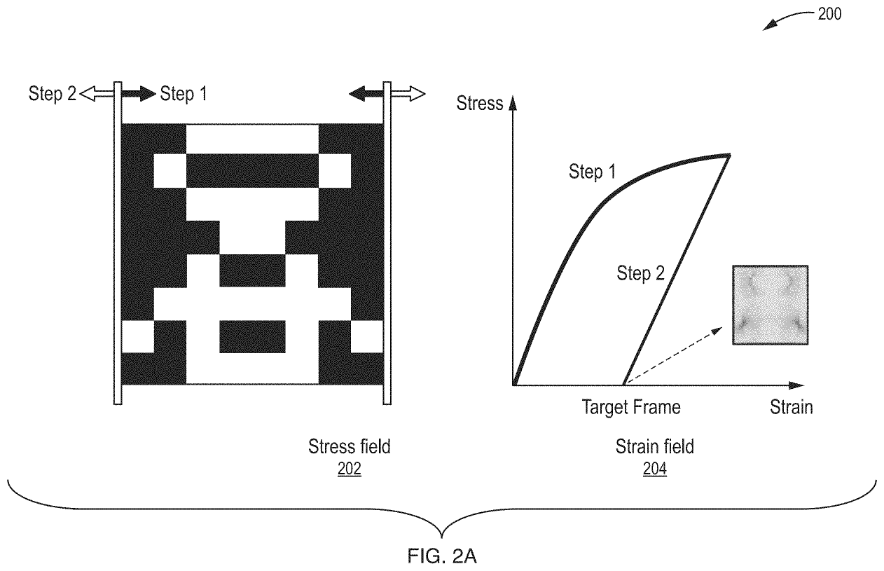

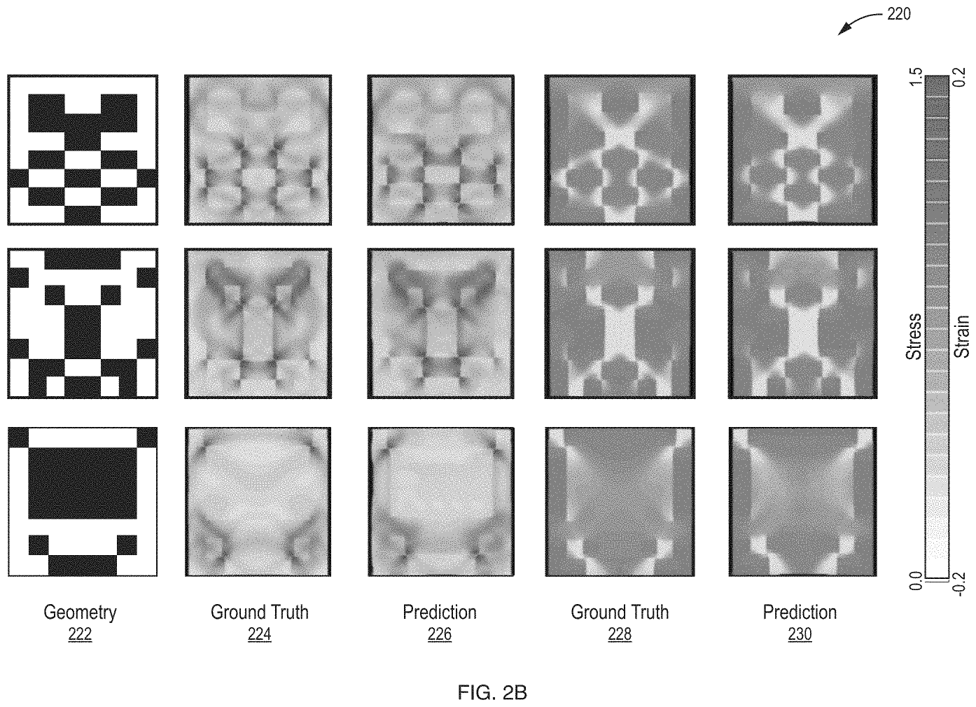

When evaluating the performance of ML models, the method calculates the L2 norm or the L2 norm map between the ground truth 484 and prediction 486 (“predicted contour 490”) of a geometry 482. The ground truth 484 is also compared with two field images for direct reference. These two field images are labeled as “random contour” 492 and “clean contour” 488 Extended Data FIG. 2). “Random contour” 492 is a contour image randomly selected from test dataset. “Clean contour” 488 is a contour image without any deformation and field (all values across the image is 0).

In embodiments, the ML calculations are performed using TensorFlow, a general-purpose ML framework. A Generative Adversarial Network (GAN) is implemented for translating composite geometries to mechanical fields. GAN is a type of deep neural network consisting of a Generator and a Discriminator based on game theory. Particularly in the model of the present disclosure, U-Net is the Generator and PatchGAN is the Discriminator. U-Net is a deep neural network model that has been used for biomedical image segmentation in earlier work. In embodiments, U-Net is employed to generate fake field images based on composite geometries. PatchGAN evaluates the generated field images from U-Net by comparing with real field images. The model was trained using a single NVIDIA Quadro RTX 4000 with 8 GB memory. The numbers of training epochs range from 150 to 400 in different cases to achieve convergence.

The Generator U-Net consists of two components encoder and decoder with skip connections between mirrored layers. Both encoder and decoder contain layers including input and output layers. The complete architecture is illustrated by Table 4, below. The trainable number of parameters in total is approximately 5,000,000. The Generator loss function is defined as:

gen_loss=gan_loss+LAMBDA×L1_loss

Here, gan_loss a binary cross entropy loss of the generated images and an array of ones. L1_loss is mean absolute error) between the generated image and the target image. LAMBDA is proportion coefficient equal to 100. In embodiments, a sigmoid activation function can also be applied.

TABLE 4
Generator UNet Architecture.

| Layer # | Layer type | Output shape |
|---|---|---|
| 1 | Input layer | (256, 256, 3) |
| 2 | Conv2D + BatchNomalization + LeakyRuLU | (128, 128, 64) |
| 3 | Conv2D + BatchNomalization + LeakyRuLU | (64, 64, 128) |
| 4 | Conv2D + BatchNomalization + LeakyRuLU | (32, 32, 256) |
| 5 | Conv2D + BatchNomalization + LeakyRuLU | (16, 16, 512) |
| 6 | Conv2D + BatchNomalization + LeakyRuLU | (8, 8, 512) |
| 7 | Conv2D + BatchNomalization + LeakyRuLU | (4, 4, 512) |
| 8 | Conv2D + BatchNomalization + LeakyRuLU | (2, 2, 512) |
| 9 | Conv2D + BatchNomalization + LeakyRuLU | (1, 1, 512) |
| 10 | Conv2D_transpose + BatchNomalization + | (2, 2, 1024) |
| | Dropout | |
| 11 | Conv2D_transpose + BatchNomalization + | (4, 4, 1024) |
| | Dropout | |
| 12 | Conv2D_transpose + BatchNomalization + | (8, 8, 1024) |
| | Dropout | |
| 13 | Conv2D_transpose + BatchNomalization | (16, 16, 1024) |
| 14 | Conv2D_transpose + BatchNomalization | (32, 32, 1024) |
| 15 | Conv2D_transpose + BatchNomalization | (64, 64, 256) |
| 16 | Conv2D_transpose + BatchNomalization | (128, 128, 128) |
| 17 | Output layer | (256, 256, 3) |

The Discriminator PatchGAN includes eight layers with approximately 3,000,000 trainable weights. The complete architecture is shown in Table 3, below. PatchGAN evaluates the generated field images by classifying individual patches (e.g., slicing the image into 30×30 patches in one embodiment) in the image as “real” or “fake”. The Discriminator loss function is defined as:

gen_loss=real_loss+generated_loss

Here, real_loss is a sigmoid cross entropy loss of real images and an array of ones, generated_loss is a sigmoid cross entropy loss of the generated images and an array of zeros.

TABLE 3
Discriminator PatchGAN architecture.

| Layer # | Layer type | Output shape |
|---|---|---|
| 1 | Concatenate layer | (256, 256, 6) |
| 2 | Conv2D + LeakyRuLU | (128, 128, 64) |
| 3 | Conv2D + BatchNomalization + | (64, 64, 128) |
| | LeakyRuLU | |
| 4 | Conv2D + BatchNomalization + | (32, 32, 256) |
| | LeakyRuLU | |
| 5 | ZeroPadding2D | (34, 34, 256) |
| 6 | Conv2D + BatchNomalization + | (31, 31, 512) |
| | LeakyRuLU | |
| 7 | ZeroPadding2D | (33, 33, 512) |
| 8 | Conv2D | (30, 30, 1) |

The disclosed method can also process non-square features. Hexagonal and triangular representations are employed to test whether the model can be applied to composites with different shapes. The sizes of composites in hexagonal and triangular representations are 312×315 and 320×332 (xxy). At the boundaries of composite, both hexagonal and triangular units are cut half. The field images for both cases were generated after one-cycle compressive loading and unloading. The magnitude of compressive strain is 10% for the hexagonal representation and 5% for the triangular representation. A smaller strain is employed for triangular cases to avoid singularity at the edges of triangles when FEM simulations are carried out.

Many distinct loading conditions can be employed with the present method. To explore the capacity of the ML model to predict strain or stress fields under various loads, two mechanical tests, nanoindentation and compression, are utilized. The general setup of those mechanical tests using FEM is same as the setup mentioned above. However, the method does not unload the composite after loading. The reason is that the field image after unloading in nanoindentation contains little information as the composite has most of its region stress-free. A compressive strain with magnitude of 5% is used again to avoid singularity during the nanoindentation simulation. In an embodiment, green lines are employed to represent analytical rigid bodies which induce deformation to include loading condition in images.

As for the nanoindentation test, rather than a straight-line rigid body, a spherical indenter is employed to implement loading. The indenter is laid at the top center of the composite with radius of 40 (unit consistent with Table 1, above). In addition to the boundary condition disclosed above, the motion at the bottom of the composite is disabled along the y-axis direction for both compression and nanoindentation. With consistent boundary conditions, the method can combine more than one loads in one mechanical test.

To capture more complex patterns, the method generates additional composite designs with high-resolution geometry (e.g., 32×32) adds the composite designs and adds the composite designs to the dataset. Keeping the length of composites consistent, smaller square units with lengths of 10 are used. All settings for FEM are the same as those considering low-resolution geometry (e.g., 8×8), except for two minor differences. First, the element size decreases from six to two for calculation accuracy and convergence. Second, the magnitude of compressive strain is set to 5% instead of 10%. The reason to use a smaller strain is to avoid singularity when running FEM as the size of soft/brittle units is smaller.

For the GAN model, the method employs original images with size of 512×512 as the input as the geometries are more complicated with smaller block units. As a consequence, one extra convolutional layer is added ahead of all layers in Generator to downsample images from 512×512 to 256×256, in embodiments. Similarly, there is one more layer after all layers in Discriminator to “upsample” images from 256×256 to 512×512, in embodiments.

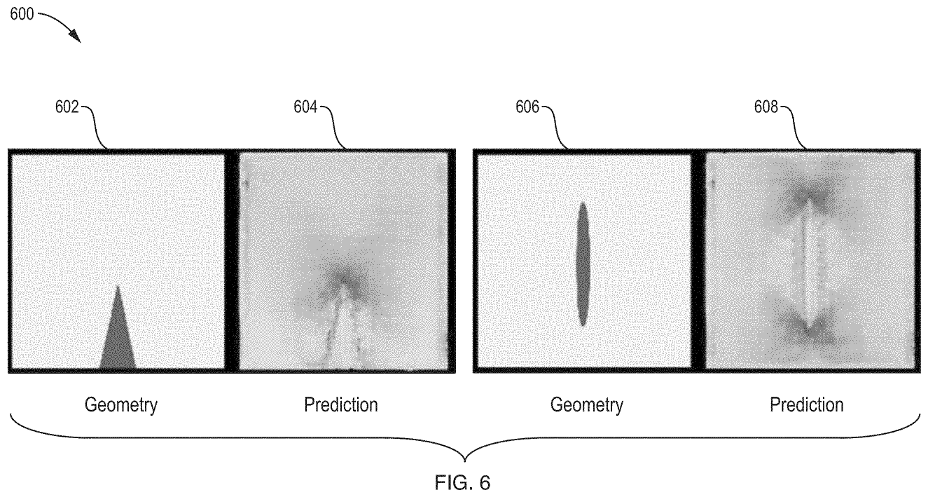

Soft materials act like cracks when being surrounded by brittle materials as they create large stress concentration and take majority of deformations. Based on this idea, the method generates composites with soft materials in narrow shapes and sharp edges embedded in brittle materials. The method employs the model that is trained for high-resolution geometry to predict the stress field around soft materials. FIG. 6 is a diagram 600 illustrating two types of geometries having cracks 602 and 606 and resulting stress field predictions 604 and 608, respectively.

In order to evaluate model performance, the mechanical behaviors of composites under compressive loading and unloading (loading applied in the x-direction, or horizontal axis,) are considered. FIG. 2A is a diagram 200 illustrating an example of such an evaluation, with loading applied in the x-direction. FIG. 2A illustrates a stress field 202 and a strain field 204. The geometry image is the input while the field image of residual stress after unloading is the target. This represents a complex physical scenario, and serves as a test case to assess the method reported here.

FIG. 2B is a set of results 220 illustrating typical results of strain or stress fields predictions of the test dataset. The results are based on geometries 222 having a respective stress field ground truth 224, strain field ground truth 228, stress field prediction 226 and stress field prediction 230.

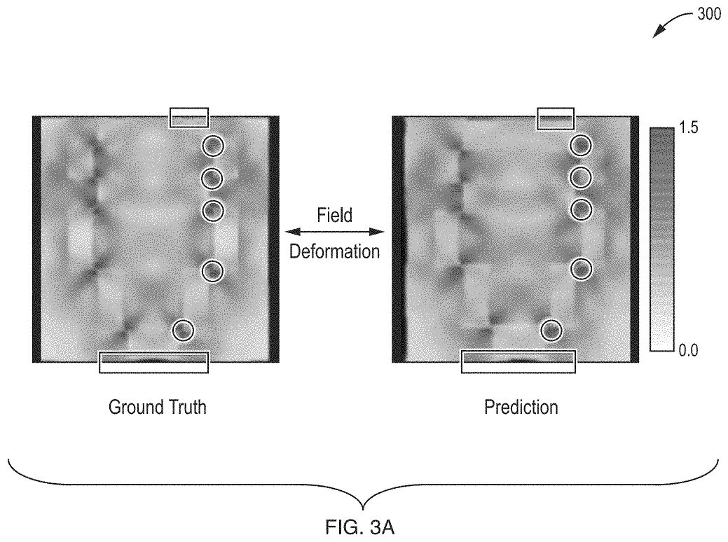

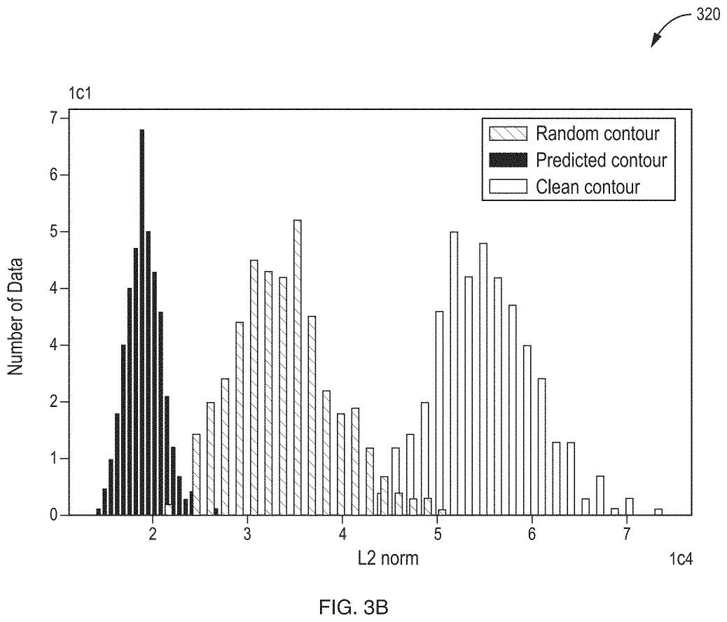

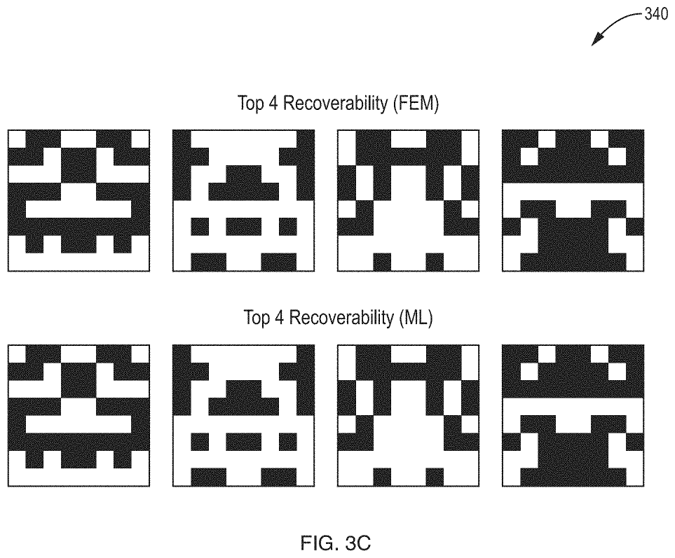

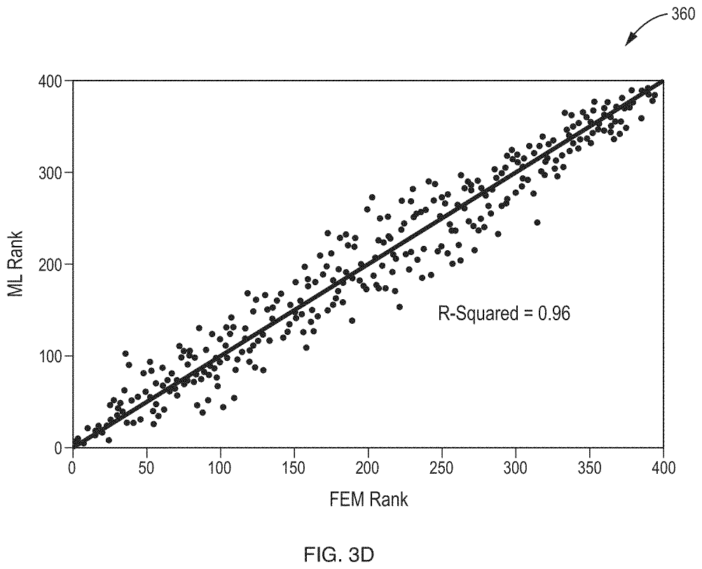

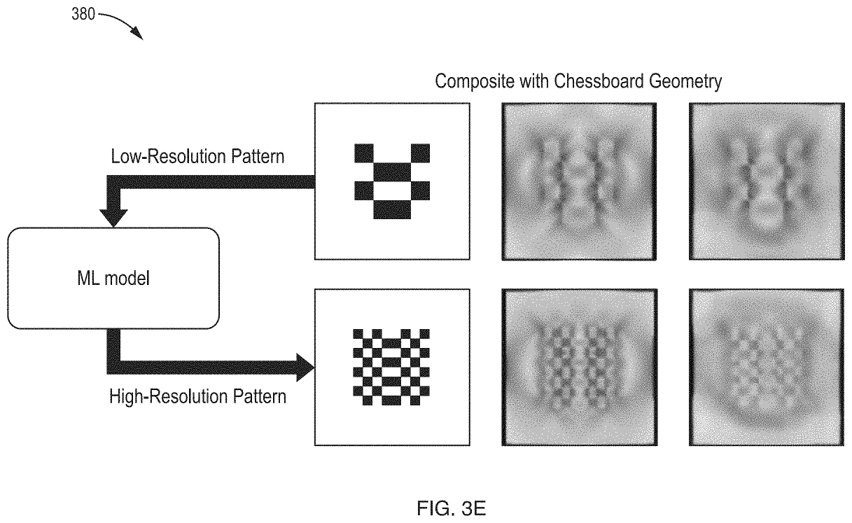

FIG. 3A is a diagram 300 illustrating stress and strain fields as well as displacements (incorporated in the overall deformation of the geometry) that are captured in the prediction. To further quantitatively evaluate the similarity between the ground truth and the prediction, the L2 norm is calculated for all 400 data in the test dataset, considering “von Mises” stress field prediction.

FIG. 3B is a graph 320 illustrating the distributions of L2 norm of predicted contours are plotted against two reference contours, clean contours and random contours. According to FIG. 3B, the mean value of the L2 norm of predicted contours is much lower when comparing with random contours (˜1.8, normalized by predicted contour) and clean contours (˜2.9, normalized by predicted contour). Furthermore, a narrower peak of predicted contours indicates that the variation is smaller as well (random contour ˜2.8, clean contour ˜2.7, normalized by predicted contour). The results reveal that the ML model features both high accuracy and applicability in predicting strain or stress fields directly from the composite geometry.

FIG. 3B illustrates two reference contour images, known as clean contour and random contour, that are selected in a similar manner as for the 8×8 representation. The L2 norm map of the clean contour exhibits the positions of stress concentration while for the random contour, the map includes mixed information of two fields. According to the L2 norm map, the predicted contour is globally accurate with low values of L2 norm across the map. For those points with large stress concentration in the image, the model shows less accuracy, because the ML model is inclined to smooth the sharp peaks—stress concentrations—to achieve an overall low loss. However, the tendency of displaying large stress in those points are clearly predicted by the model, as shown by comparing with the L2 norm map of the clean contour. With the capacity of predicting fields for high-resolution geometries, the model can be utilized to investigate composites with complex patterns as is necessary in design applications.

FIG. 3C is a diagram 340 illustrating recoverability of FEM and ML methods. With predicted fields, the method can further derive secondary mechanical properties of the composites. For instance, the method can compute mechanical recoverability of the composite after being subjected to compressive loading and unloading. Here, recoverability is defined as average residual stress of the composites. The model can predict the geometries associated with the top 4 recoverability levels in the test dataset exactly, with the information obtained from the field images.

FIG. 3D is a graph 360 illustrating ranks of recoverability of 400 composites in a test data set. Broadly, the ML model performed an accurate prediction on the ranks of recoverability over all 400 composites in the test dataset as illustrated by FIG. 3(d). The R-square value of the linear fitting between ML model prediction and the ground truth is 0.96. However, the ML model was not trained directly for predicting recoverability, as this measure is a secondary extracted feature. However, the model is still able to predict the secondary information obtained from field images precisely with only 1,600 training data. Due to recoverability, the model not only generates field images that look similar to the ground truth, but also predicts specific pixel values at high accuracy. As a consequence, the model can be used to optimize mechanical properties of composites by varying geometries. Furthermore, there is no longer a need to develop different ML models for different mechanical properties as multiple properties can be derived from predicted fields and deformation already included in the model.

FIG. 3E is a diagram 380 illustrating the method being used for local, smaller-scale patterns. In addition to mechanical property predictions, the method checks the reliability for local, smaller-scale patterns. To assess this capability, a ML model trained, in embodiments, with 8×8 representation is used to study high-resolution patterns at an increased 16×16 resolution. Clear boundaries of chessboard pattern are predicted even if the 16×16 representation has never been seen by the model. The consistence across different scales reveals that the GAN has learned a physical understanding of mechanical phenomena, in this case, stress concentrations and how they emerge from complex microstructural patterns. The model has a capacity for recognizing scale independent local patterns can be applied to predict strain or stress field of hierarchical structures and is useful for design applications that explore a broad range of de nova microstructures.

In the earlier examples the composite was composed of square units. However, the model can be extended to investigate composites with non-square elementary design units. To demonstrate such possibility, designing composite elements employs both hexagonal and triangular tessellations. To do this, the same model trained on square representation is now trained with a dataset in hexagonal and triangular representations, respectively. FIG. 4A is a diagram 400 illustrating the von Mises stress fields calculated by the FEM and the trained model show high similarity, as visualized in two example cases of triangle 402 and hexagon 412. The results indicate that the model can be easily generalized to composites of different shapes, producing a prediction 408 based on a geometry 404 and ground truth 406 for the triangle 402 example and a prediction 418 based on a geometry 414 and ground truth 416 for the hexagon example.

The input geometry images disclosed above do not yet include any information about loading conditions, which were encoded in the training. The method can be extended to train the model on multiple loading conditions, such as variations in boundary conditions, which are embedded directly in the images. The loading conditions are specified by adding green lines the geometry images and as illustrated by field images in FIG. 4A. The green lines represent rigid bodies that exert loads during simulations of FEM. The straight green lines are two rigid lines used to compress the composites along the x-axis direction. The circular green line is a spherical (2D) rigid indenter used for nanoindentation. Hence, the dataset consists of two sets of data: One set of images containing two straight green lines showing the results of compression and the other set containing one circular line exhibiting the results of nanoindentation. The geometries of composites in the two sets are the same.

FIG. 4B is a diagram 420 illustrating different loading conditions. After being trained with the mixed dataset, the model can recognize different loading conditions and make relevant predictions, as illustrated by FIG. 4B. More specifically, the model transfers loading conditions from input geometry images to the prediction and predicts the von Mises stress field according to the specified loading conditions. More surprisingly, when two or more loading conditions are combined in the geometry image, which is not included in the training dataset, the ML model is still able to predict the stress field accurately. As illustrated by FIG. 4B, the model predicts both stress fields caused by compression (e.g., the general stress pattern similar to single compression) and nanoindentation (e.g., stress concentration on the top).

The ability of the model to read two distinct loading conditions from images and predict fields accordingly shows the potential of applying one model to predict multiple mechanical tests, and offers evidence for transferability. With the well-trained model, FEM simulations that are usually carried out by conventional code can be performed at much higher speed and lower computational costs. Furthermore, the model can also be employed for complex loading conditions due to its capacity of predicting fields with mixed loading conditions.

FIG. 4C is a diagram 460 illustrating an example of a 32×32 profile 462. Geometries of real-world composites can be complicated and require high resolutions. As a consequence, a low resolution 8×8 representation may not be sufficient for practical applications. To study complex geometries, the method trains an even deeper model with 32×32 representation as illustrated by FIG. 4C. Similarly, as before, the loading condition is a one-cycle compressive loading and unloading with von Mises stress field images being the target outputs. To show an example of a more complicated composition and the associated prediction, a geometry image 466 in the shape of the MIT dome is used as an example input. Both the ground truth 468 and the field image 470 predicted by the model exhibit a similar stress pattern 464. The example reveals the potential of the model to predict high-resolution fields for general inputs.

To quantitatively investigate the accuracy of high-resolution prediction, the method calculates the L2 norm and the 8×8 representation. However, when using a 32×32 representation, there are much more local patterns than in the 8×8 representation. As a result, a comprehensive comparison of differences across the whole image is more suitable than a single value. Hence, an L2 norm map is employed instead of the L2 norm. The L2 norm map is obtained by calculating local L2 norm region by region when comparing two images. The method randomly generates a 32×32 geometry image to check the similarity between the ground truth and prediction using L2 norm map.

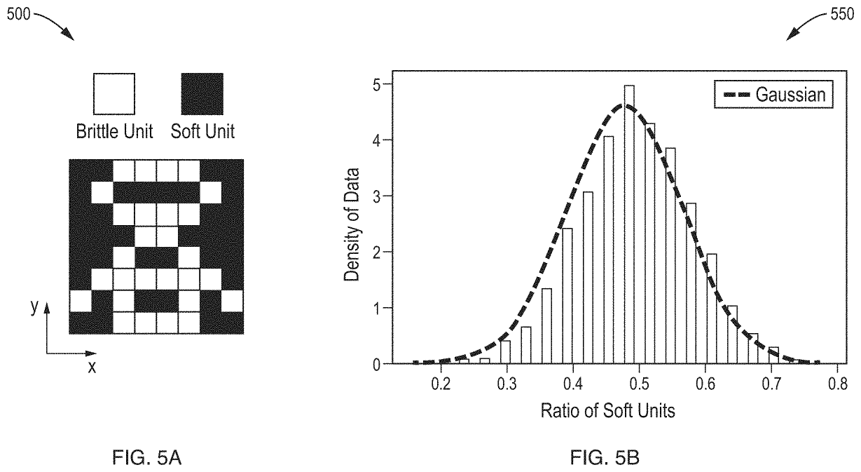

FIGS. 5A-B are diagrams of the graphical representation of composites and the distribution of 2000 data considering the ratio of soft units. The dotted line is a fitted curve of the Gaussian distribution. According to the graphical representation of the embodiment illustrated by FIGS. 5A-B, there are 2³² possible combinations in total. Among all potential geometries, the random geometry generator 102 randomly selects 2,000 data to constitute the dataset. The distribution of all 2,000 data is plotted over the ratio of soft units. As FIGS. 5A-B illustrate, the distribution generally fits a Gaussian curve with the mean value being approximately 0.5. The curve reveals a uniformly distributed dataset without skewed features. Therefore, the dataset is representative of all possible data in the search space. With 2,000 data randomly generated, the method further splits them into training dataset with 80% data and test dataset with the rest 20% data. The split is also stochastic, thus conserving the distribution.

The method can convert inputted geometry to a strain field or stress field. In an example embodiment, 2D composites with two constituent materials, defined as soft units (illustrated in FIG. 5A as red) and brittle units (illustrated in FIG. 5B as white), can be examined. Both soft and brittle units have linear plasticity and strain hardening, which is characterized by the “crushable foam” model in simulators such as Abaqus. Brittle units have relatively larger Young's modulus (e.g., the property that represents how easily a material can stretch and deformed, defined by the ratio of tensile stress to tensile strain) but smaller yield strain comparing with soft units. In this example, the initial composite considered includes 8×8 block units so that a brute-force method can validate the ML method against full-physics simulations. While the generated patterns are at coarse resolutions (8×8) in the first example), the overall image resolution is 256×256 pixels. A person of ordinary skill in the art can recognize that resolution can be increased to higher amounts.

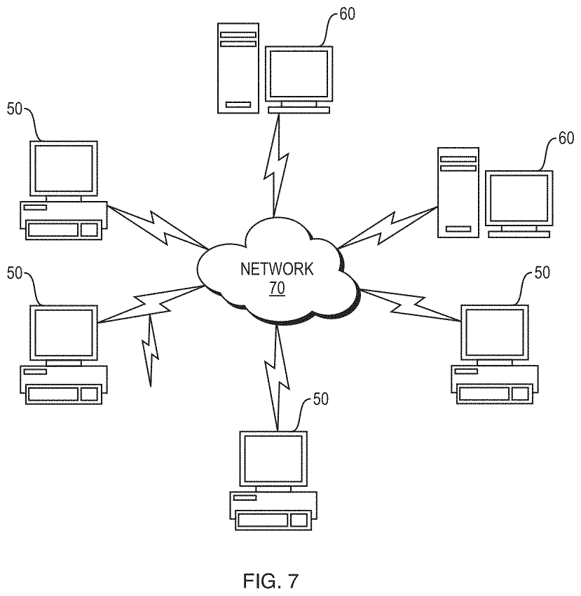

FIG. 7 illustrates a computer network or similar digital processing environment in which embodiments of the present invention may be implemented.

Client computer(s)/devices 50 and server computer(s) 60 provide processing, storage, and input/output devices executing application programs and the like. The client computer(s)/devices 50 can also be linked through communications network 70 to other computing devices, including other client devices/processes 50 and server computer(s) 60. The communications network 70 can be part of a remote access network, a global network (e.g., the Internet), a worldwide collection of computers, local area or wide area networks, and gateways that currently use respective protocols (TCP/IP, Bluetooth®, etc.) to communicate with one another. Other electronic device/computer network architectures are suitable.

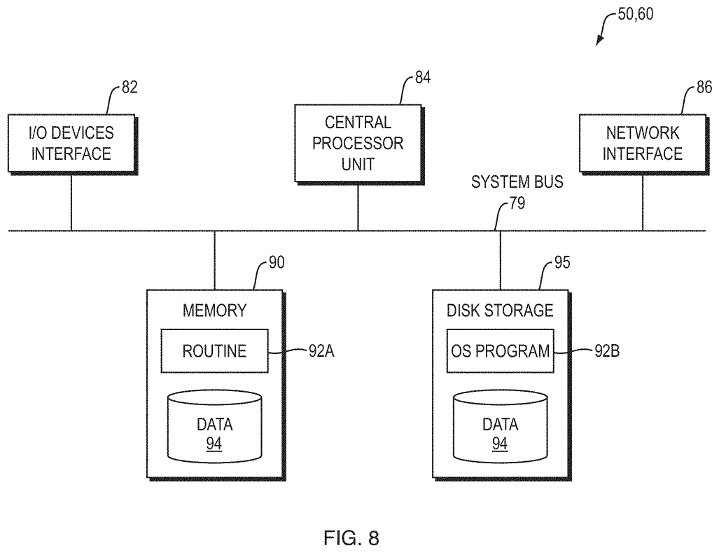

FIG. 8 is a diagram of an example internal structure of a computer (e.g., client processor/device 50 or server computers 60) in the computer system of FIG. 7. Each computer 50, 60 contains a system bus 79, where a bus is a set of hardware lines used for data transfer among the components of a computer or processing system. The system bus 79 is essentially a shared conduit that connects different elements of a computer system (e.g., processor, disk storage, memory, input/output ports, network ports, etc.) that enables the transfer of information between the elements. Attached to the system bus 79 is an I/O device interface 82 for connecting various input and output devices (e.g., keyboard, mouse, displays, printers, speakers, etc.) to the computer 50, 60. A network interface 86 allows the computer to connect to various other devices attached to a network (e.g., network 70 of FIG. 5). Memory 90 provides volatile storage for computer software instructions 92 and data 94 used to implement an embodiment of the present invention (e.g., generator module, discriminator module, loss modules, generative adversarial model code detailed above). Disk storage 95 provides non-volatile storage for computer software instructions 92 and data 94 used to implement an embodiment of the present invention. A central processor unit 84 is also attached to the system bus 79 and provides for the execution of computer instructions.

In one embodiment, the processor routines 92 and data 94 are a computer program product (generally referenced 92), including a non-transitory computer-readable medium (e.g., a removable storage medium such as one or more DVD-ROM's, CD-ROM's, diskettes, tapes, etc.) that provides at least a portion of the software instructions for the invention system. The computer program product 92 can be installed by any suitable software installation procedure, as is well known in the art. In another embodiment, at least a portion of the software instructions may also be downloaded over a cable communication and/or wireless connection. In other embodiments, the invention programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals may be employed to provide at least a portion of the software instructions for the present invention routines/program 92.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method comprising:
generating, at generator neural network, a fake field image based on adding noise to an inputted geometry images of a composite;
determining, at a discriminator neural network, whether the fake image generated by the generator based on the generated geometry images represent a ground truth; and
generating a field prediction of at least one of a global property of a material and local property of the material, the field prediction being generated upon the discriminator neural network determining the fake image generated represent the ground truth, the field prediction being a prediction of at least one of a stress field and a strain field of the generated geometry images.

2. The method of claim 1, further comprising:
repeating the generating and determining until the discriminator determines the fake image generated by the generator represents the ground truth.

3. The method of claim 1, further comprising:
comparing the field prediction of the composites to a field prediction of strain and stress field information generated using a finite element method (FEM) to determine accuracy of the field prediction.

4. The method of claim 1, wherein the geometry images encode material composition and boundary conditions.

5. The method of claim 1, wherein the geometry image includes microstructure.

6. The method of claim 1, wherein the geometry image includes brittle units and soft units, the brittle units and soft unit being mechanically distinct in the properties of elasticity and plasticity.

7. The method of claim 1, further comprising:
based on the field prediction, translating a three-dimensional model into an additive manufacturing model for three-dimensional printing.

8. The method of claim 1, wherein the generator and discriminator form a machine learning network.

9. The method of claim 8, wherein the machine learning network is a generative adversarial network (GAN).

10. A method comprising:
training a machine learning network having a generator and a discriminator by:
generating, at the generator, field images having random noises added based on inputted geometric images, the generator having a training objective to increase an error rate of the discriminator; and
comparing, using the discriminator, the generated field images to real field images, each comparison determining whether the field images from the generator are real or fake, the discriminator having a training objective to optimize a capacity of identifying fake images produced by the generator;
wherein the machine learning network is trained when the discriminator and generator reach an equilibrium, the equilibrium being one of the component networks maintaining its status regardless of the other component networks.

11. The method of claim 10, further comprising:
randomly generating the geometry images of the composites to be inputted to the generator.

12. The method of claim 10, further comprising:
analyzing the randomly generated geometry images of the composites using a finite element method (FEM) to obtain real strain and stress field information to provide to the discriminator.

13. The method of claim 10, wherein the geometry images encode material composition and boundary conditions.

14. The method of claim 10, wherein the geometry image includes microstructure.

15. The method of claim 10, wherein the geometry image includes brittle units and soft units, the brittle units and soft unit being mechanically distinct in the properties of elasticity and plasticity.

16. The method of claim 10, further comprising:
running the trained machine learning network by providing a particular geometry image to the machine learning network, thereby generating a field prediction of stress fields and strain field of the particular geometry image; and
based on the field prediction, translating a three-dimensional model into an additive manufacturing model for three-dimensional printing.

17. A system comprising:
a processor; and
a memory with computer code instructions stored thereon, the processor and the memory, with the computer code instructions, being configured to cause the system to:
train a machine learning network having a generator and a discriminator by:
generating, at the generator, field images having random noises added based on inputted geometric images, the generator having a training objective to increase an error rate of the discriminator; and
comparing, using the discriminator, the generated field images to real field images, each comparison determining whether the field images from the generator are real or fake, the discriminator having a training objective to optimize a capacity of identifying fake images produced by the generator;
wherein the machine learning network is trained when the discriminator and generator reach an equilibrium, the equilibrium being one of the component networks maintaining its status regardless of the other component networks.

18. The system of claim 17, wherein the instructions are further configured to cause the system to:
randomly generating the geometry images of the composites to be inputted to the generator.

19. The system of claim 17, wherein the instructions are further configured to cause the system to:
analyzing the randomly generated geometry images of the composites using a finite element method (FEM) to obtain real strain and stress field information to provide to the discriminator.

20. The system of claim 17, wherein the geometry images encode material composition and boundary conditions.
